(19) United States
(12) Patent Application Publication
(10) Pub. No.: US 2015/0064706 A1
(43) Pub. Date: Mar. 5, 2015

(54) Detection and Mixing in a Conduit in Integrated Bioanalysis Systems

(71) Applicant: APPLIED BIOSYSTEMS, LLC, Carlsbad, CA (US)

(72) Inventor: Charles S. VANN, El Granada, CA (US)

(21) Appl. No.: 14/453,494

(22) Filed: Aug. 6, 2014

Related U.S. Application Data
(63) Continuation of application No. 14/256,832, filed on Apr. 18, 2014.
Application No. 14/256,832 is a continuation of application No. 14/150,577, filed on Jan. 8, 2014, now abandoned.
Application No. 14/150,577 is a continuation of application No. 14/026,997, filed on Sep. 13, 2013, now abandoned.
Application No. 14/026,997 is a continuation of application No. 12/165,551, filed on Jun. 30, 2008, now abandoned.
(60) Provisional application No. 60/946,950, filed on Jun. 28, 2007.

(51) Int. Cl. C12Q 1/68; B01L 3/00; G01N 21/64; B01L 7/00; G01N 21/76

(52) U.S. Cl. CPC C12Q 1/686; B01L 7/525; G01N 21/76; G01N 21/64; B01L 3/502784; B01L 2300/0832; B01L 2300/0654; B01L 2400/0478; B01L 2200/0673; B01L 2300/1805
USPC 435/6.12; 435/287.2

(57) ABSTRACT

Apparatuses and methods in which detection is integrated with various liquid processing and environmental control functions to create integrated bioanalysis systems are disclosed. Though the various integrated bioanalysis systems are useful for any number of analysis formats, they are adaptable to high-throughput processing of samples.

### CROSS-REFERENCE TO RELATED APPLICATIONS

This application claims a priority benefit under 35 U.S.C. §119(e) from U.S. Patent Application No. 60/946,950, filed Jun. 28, 2007, which is incorporated herein by reference.

### FIELD

The field of the present disclosure relates to apparatuses and methods for high-throughput detection in integrated bioanalysis systems.

### BACKGROUND

Generally, in bioanalysis, liquid processing is essential for the many process steps involved in obtaining a result. Additionally, many analysis steps, such as sample preparation, reaction, separation, detection, and data processing involved in a broad range of bioanalyses usually require a variety of devices and instrumentation.

For many types of bioanalyses, there is desire to reduce the physical complexity of the biotechnology laboratory and at the same time increase throughput. Therefore, there is a need in the art for bioanalysis systems that can integrate analysis steps such as sample preparation, reaction, separation, detection, and data processing into a single footprint, and at the same time have the flexibility to scale throughput.

All patents, applications, and publications mentioned here and throughout the application are incorporated in their entireties by reference herein and form a part of the present application.

### BRIEF DESCRIPTION OF THE DRAWINGS

FIG. 1A and FIG. 1B depict variations of liquid processing manifolds for use in embodiments of integrated bioanalysis systems.

FIG. 2A is a perspective view depicting an integrated bioanalysis system illustrative of the present teachings, and FIG. 2B is a cross-section of a side view depicting a subassembly of FIG. 2A.

FIG. 3A and FIG. 3B are perspective views that depict variations of integrated bioanalysis systems illustrative of the present teachings.

FIG. 4A is a perspective view depicting an integrated bioanalysis system illustrative of the present teachings, and FIG. 4B is a cross-section of a side view depicting a subassembly of FIG. 4A.

FIG. 5 depicts a variation of a scanning detection device for use in conjunction with embodiments of liquid processing manifolds.

FIGS. 6A-6C depict a method for mixing two liquids using various embodiments of liquid processing manifolds, illustrative of the present teachings.

FIGS. 7A-7C depict a method for mixing two liquids using various embodiments of liquid processing manifolds illustrative of the present teachings.

It is to be understood that the figures are not drawn to scale, nor are the objects in the figures necessarily drawn to scale in relationship to one another. The figures are depictions that are intended to bring clarity and understanding to various embodiments of apparatuses and methods disclosed herein. Wherever possible, the same reference numbers will be used throughout the drawings to refer to the same or like parts.

### DETAILED DESCRIPTION

What is disclosed herein are various embodiments of apparatuses and methods in which luminescent detection is integrated with various analysis steps that are practiced in a range of biological analyses. In bioanalysis, functions such as sample preparation, reaction, and separation require the processing of fluids, such as, for example, the dispensing, mixing, and transport of liquids. Additionally, control of environmental conditions that impact analysis, such as, for example, temperature, pH, and ionic strength is frequently required. In the various embodiments of apparatuses and methods disclosed herein, detection is integrated with various liquid processing and environmental control functions to create integrated bioanalysis systems thereby. Though the various embodiments of integrated bioanalysis systems are useful for any number of analysis formats, they are adaptable to high-throughput processing of samples.

In disclosed embodiments of apparatuses and methods for integrated bioanalysis systems, liquid processing, environmental control and detection are integrated functions that can be performed in individual conduits. In various embodiments, a plurality of conduits comprises a liquid processing manifold.

The term “conduit” as used herein is any number of liquid processing components known in the art of bioanalysis, such as, but not limited by, tubing, piping, needle, pipette, and pipette tip. Such conduits are useful in a variety of manipulations of samples and reagents for a variety of bioanalyses.

The term “luminescent detection” as used herein includes photoluminescent detection, such as fluorescence and phosphorescence, as well as chemiluminescent detection, including bioluminescent detection. These types of luminescent detection are useful for a wide range of bioanalyses, offering sensitive detection over a wide range of analytes such as nucleic acids, polypeptides, hormones, drug substances, and the like. An exemplary class of bioanalyses are enabled by a technique know as the polymerase chain reaction (PCR). Some examples of bioanalyses that utilize the PCR technique include viral quantitation, quantitation of gene expression, drug therapy efficacy, DNA damage measurement, pathogen detection, and genotyping.

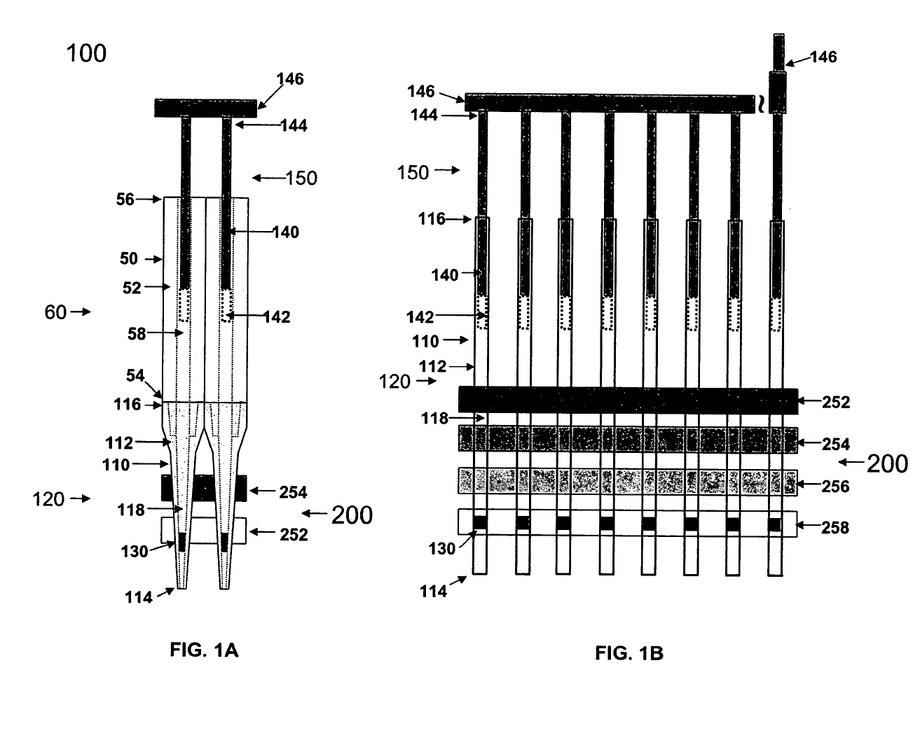

As previously mentioned, in various embodiments of apparatuses and methods for integrated bioanalysis systems, liquid processing, environmental control and detection are integrated functions that can be performed in individual conduits. Additionally, a liquid processing manifold including a plurality of conduits can be useful for high throughput liquid processing systems. The various embodiments of liquid processing manifold 100 depicted in FIG. 1A and FIG. 1B can be used with embodiments of integrated bioanalysis systems. Liquid processing manifold 100 of FIG. 1A and FIG. 1B can have a conduit assembly 120 having a plurality of conduits 110. In various embodiments of liquid processing manifold 100 of FIG. 1A and FIG. 1B, conduit 110 can be removable and replaceable. Conduit 110 has a body 112 which has a first end 114 and a second end 116, and has a bore 118 extending through body 112. In some embodiments of liquid processing manifold 100, conduit assembly 120 can have conduits 110 that are arranged in a linear array. In some embodiments of liquid processing manifold 100, conduit assembly 120 can have conduits 110 that can be arranged in numerous types of two-dimensional geometries. In some embodiments of liquid processing manifold 100, conduit 110 can be fabricated from a polymeric material, for example, but not limited by, from classes of polymers such as polypropylene, polyethylene, polyhalohydrocarbon, polycarbonates, and polysilicones, and combinations thereof. In some embodiments of liquid processing manifold 100, conduit 110 can be fabricated from an inorganic oxide material, for example, but not limited by such as quartz, fused silica, and sapphire, and combinations thereof. In some embodiments of liquid processing manifold 100, conduit 110 can be fabricated from a metal, such as but not limited by stainless steel, titanium, and combinations thereof. In such embodiments, the metal may be lined with a polymer or inorganic oxide material. In general, attributes for conduits 100 of conduit assembly 120 include, but are not limited by, chemical, mechanical, and thermal stability for their intended use in bioanalysis.

In addition to conduit assembly 120, various embodiments of liquid processing manifold 100 of FIG. 1A and FIG. 1B can have a plunger or piston assembly 150 to provide control for processing fluids. In some embodiments of liquid processing manifold 100 of FIG. 1A, piston assembly 150 can function in a housing assembly 60, that can have a plurality of piston housings 50. Piston housing 50 has a body 52 having first end 54 and a second end 56, with a bore 58 extending through body 52. The second end 116 of conduit 110 can be fitted to first end 54 of the piston housing 50 so that piston housing bore 58 is in fluid communication with conduit bore 118. Piston assembly 150 can have a plurality of pistons 140. Piston 140 has a first end 142, which sealably engages piston housing bore 58 and conduit bore 118, and a second end 144, which can be connected to mechanical means for moving the piston 140, depicted by bar 146 in FIG. 1A and FIG. 1B. As indicated in FIG. 1B, which shows two variations for bar 146, mechanical means for moving the piston 140 can be fashioned to move a plurality of pistons, or to move them individually. In FIG. 1A and 1B, conduit 110 has first end 114 in which a liquid aliquot or slug 130 can be processed using various embodiments of liquid processing manifolds 100.

In various embodiments of liquid processing manifold 100 of FIG. 1A and 1B, the movement of piston 140 causes a displacement of fluid in conduit 110, controlling the movement of fluids in conduit 110 thereby. Such control of fluids may be useful for many types of manipulations of fluids, such as, but not limited by aspiration, mixing, aliquoting, and dispensing, and the like. Moreover, various embodiments of liquid processing manifold 100 enable the processing of a few samples for low throughput processing or many samples for high throughput processing.

In some bioanalyses, piston housing bore 58 and conduit bore 118 of FIG. 1A and 18 may be other than an air/liquid interface for manipulating a liquid aliquot or slug 130 in order to provide an interface tension greater than that provided by an air/liquid interface. In some embodiments of liquid processing manifold 100, first end 142 of piston 140 may come in direct contact with liquid aliquot or slug 130 to provide a solid/liquid interface. In some variations of liquid processing manifold 100, the bore-space between first end 142 of piston 140 and liquid aliquot or slug 130 may be partially or totally filled with a fluid that is inert and immiscible and in contact with liquid aliquot or slug 130, providing a liquid/liquid interface thereby. For example, since the vast majority of bioanalyses are aqueous-based, an example of such an inert, immiscible fluid can be an oil, such as a mineral oil. Additionally, it is desirable that the coefficient of expansion of the inert fluid be low, so as to minimize the impact of the change in volume of the inert fluid when thermostating system 200 is used in variations of integrated bioanalysis system 500.

In various embodiments of liquid processing manifold 100 of FIG. 1A and 1B, liquid aliquot or slug 130 positioned at first end 114 of conduit 110 can be finely manipulated and controlled. The phrase “positioned at first end 114” in reference to position of a liquid aliquot or slug 130 may include embodiments where liquid aliquot or slug 130 can be within the first end, and remains at a position proximal to first end 114, as well as embodiments where the liquid aliquot or slug 130 can be at least partially extended from first end 114. In some embodiments, liquid aliquot or slug 130 can be enveloped by an inert, immiscible fluid, such as an oil, for example a mineral oil, so that the protruding liquid can be an oil droplet or film. As will be discussed in more detail subsequently, liquid aliquot or slug 130 can be positioned at first end 114 so that it may be readily detected.

For various disclosed embodiments of integrated bioanalysis system 500, a thermostating system 200 can be provided to conduit assembly 120 of the liquid processing manifolds 100 by providing one or a plurality of thermostating units, such as for example, thermostating units 252 and 254 of FIG. 1A or thermostating units 252, 254, 256 and 258 of FIG. 1B. In addition to the thermostating units, such as 252 and 254 of FIG. 1A, thermostating unit 200 may include additional components, such as thermisters and controllers. The plurality of thermostating units can provide discrete thermal zones for each conduit 110, which discrete zones may be maintained at a desired temperature. In some embodiments of a thermostating system 200, the thermostating units may be for example Peltier devices, providing the capability to heat or cool the discrete thermal zones to a desired temperature. In other embodiments of a thermostating system 200 the thermostating units may be, for example, heat blocks that can heat each discrete thermal zones to a desired temperature. An example of an integration of a liquid processing manifold with thermal control for heat cycling during PCR amplification can be found in U.S. Pat. No. 5,985,651 (Hunike-Smith; Nov. 16, 1999).

Various embodiments of liquid processing manifolds 100, fitted with a thermostating system 200 may be incorporated into embodiments of integrated bioanalysis systems. Such systems are integrated to provide a complete range of liquid processing and detection adapted to conduit 110, so that in addition to liquid processing, the conduit 110 serves as a reaction and detection vessel. Various embodiments of disclosed integrated bioanalysis systems provide flexibility to the end user by providing flexibility in throughput from a few samples to many, flexibility over the volume of liquid aliquot or slug 130 processed by selection of conduit inner diameter and slug length, and flexibility over assay format through selection of automated liquid processing providing control to individual or selected numbers of conduits.

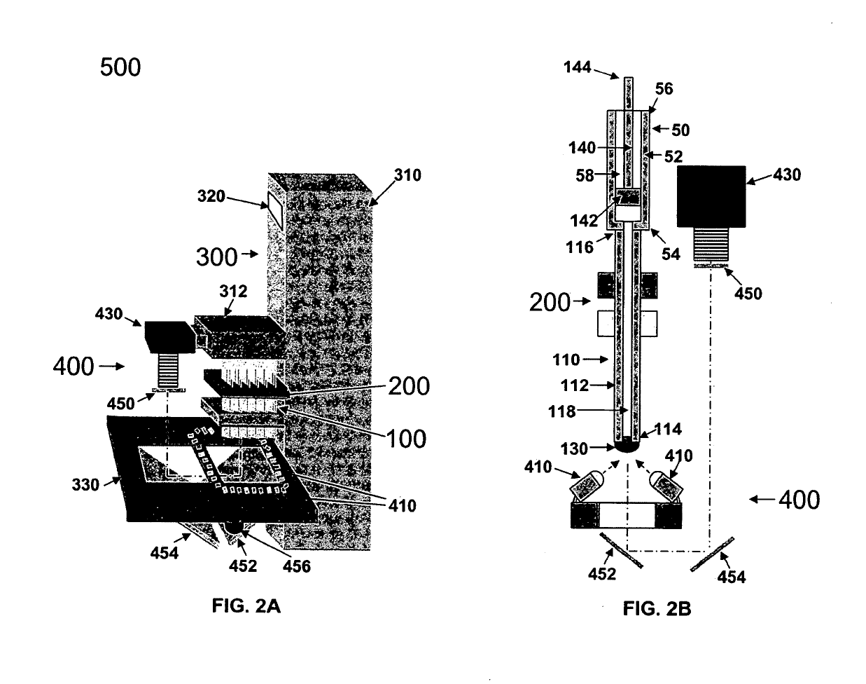

FIG. 2A is a perspective view of integrated bioanalysis system 500 according to various embodiments of the present teachings. The integrated bioanalysis system 500 can have instrument support unit 300 which includes instrument support housing 310, which can be a housing for instrument control system 320. Additionally, instrument support unit 300 can act as a mount for liquid processing manifold 100 using liquid processing manifold chassis 312, stage 330, and detection system 400. Instrument control system 320 can control the operation of liquid processing manifold 100, control thermostating system 200, as well as the control the movement of stage 330, and the operation of detection system 400. Additionally, instrument control system 320 may provide data processing and report preparation functions. All such instrument control functions may be dedicated locally to the integrated bioanalysis system 500, or instrument control system 320 may provide remote control of part or all of the control, analysis, and reporting functions.

The detection system 400 of FIG. 2A has excitation source 410, detector 430, and an optical train including filter 450, first mirror 452, second mirror 454, and motor 456 that can be used to control the position of first mirror 452 and second mirror 454. According to various embodiments, a detection system can comprise one or more excitation sources, detectors, operational amplifiers, and current control circuits. Such components may have temperature dependent properties, meaning that their properties (e.g., LED intensity) can change with temperature variations, which will be discussed in more detail subsequently. In FIG. 2A, excitation source 410 is depicted as an array of light emitting diodes (LEDs), though different embodiments of detection system 400 may use various excitation sources. An excitation source 410 is used to excite chemical or biochemical species in liquid aliquot or slug 130 positioned at first end 114 of conduit 110, which first end serves as a reaction and detection vessel. The terms “excitation source,” “irradiation source,” and “light source” are used in the art interchangeably.

The term “LED” or “light emitting diode” is used herein to refer to conventional light-emitting diodes, i.e., inorganic semiconductor diodes that convert applied electrical energy to light, as well as organic light emitting diode (OLEDs). Conventional LEDs include, for example, aluminum gallium arsenide (AlGaAs), which generally produce red and infrared light, gallium aluminum phosphide, which generally produce green light, gallium arsenide/phosphide (GaAsP), which generally produce red, orange-red, orange, and yellow light, gallium nitride, which generally produce green, pure green (or emerald green), and blue light, gallium phosphide (GaP), which generally produce red, yellow and green light, zinc selenide (ZnSe), which generally produce blue light, indium gallium nitride (InGaN), which generally produce bluish-green and blue light, indium gallium aluminum phosphide, which generally produce orange-red, orange, yellow, and green light, silicon carbide (SIC), which generally produce blue light, diamond, which generally produce ultraviolet light, and silicon (Si), which are under development. LEDs are not limited to narrowband or monochromatic light LEDs; LEDs may also include broad band, multiple band, and generally white light LEDs. Organic LEDs can be polymer-based or small-molecule-based (organic or inorganic), edge emitting diodes (ELED), Thin Film Electroluminescent Device s(TFELD), Quantum dot based inorganic “organic LEDs,” and phosphorescent OLED (PHOLED). In addition to LEDs and OLEDs, some embodiments of integrated bioanalysis system 500 may utilized excitation sources such as lasers, for example solid state lasers, such as YAG lasers, gas lasers, such as helium neon (HeNe) lasers, and diode lasers as well as lamps, such as for example, deuterium or mercury lamps.

According to some embodiments of detection system 400 of FIG. 2A of integrated bioanalysis system 500, excitation source 410 can illuminate an entire conduit assembly 120. In other embodiments, detection system 400, excitation source 410 can be directed to illuminate portions of first ends 114 of conduit assembly 120 (see FIGS. 1A and 1B). An excitation source 410 can include, for example, a combination of two, three, or more LEDs, OLEDs, laser diodes, and the like that are positioned to illuminate all or a portion of conduit assembly 120. In some embodiments, the LEDs may be white light LEDs that illuminate all or a portion of conduit assembly 120. In some embodiments, all or a portion of conduit assembly 120 may be illuminated by LEDs having a first relatively short wavelength in the visible range of the electromagnetic spectrum (e.g., UV-blue within the range of 380 nm to 495 nm), a second longer wavelength LED (e.g., green within the range of 450 nm to 495 nm), or a third longer wavelength LED (e.g., red within the range of 620 nm to 750 nm). In various embodiments, excitation source 410 of FIG. 2A that illuminates all or a portion of conduit assembly 120 may include combinations of LEDs having different wavelengths in the UV-visible range of the electromagnetic spectrum of between about 380 nm to about 750 nm.

The term “detector” refers to devices that convert electromagnetic energy into an electrical signal, and may include both single element, multi-element and array optical detectors. As previously mentioned, excitation source 410 is used to excite chemical or biochemical species in liquid aliquot or slug 130 positioned at first end 114 of conduit 110. For the phenomenon of luminescent detection, such excited chemical or biochemical species emit electromagnetic radiation of a longer wavelength than the excitation source. As such, detector 430 is a device capable of monitoring the electromagnetic (e.g., optical) signal from the chemical or biochemical species in liquid aliquot or slug 130 positioned at first end 114 of conduit 110, providing an electrical output signal or data related to the monitored electromagnetic (e.g., optical) signal. Such devices include, for example, but not limited by photodiodes, including avalanche photodiodes, phototransistors, photoconductive detectors, linear sensor arrays, CCD detectors, CMOS optical detectors (including CMOS array detectors), photomultipliers, and photomultiplier arrays. According to certain embodiments, an optical detector, such as a photodiode or photomultiplier, may contain additional signal conditioning or processing electronics. For example, an optical detector may include at least one pre-amplifier, electronic filter, or integrating circuit. Suitable preamplifiers include integrating, transimpedance, and current gain (current mirror) pre-amplifiers.

As shown in FIG. 2A, detector 430 may be mounted from liquid processing manifold chassis 312, though detector 430 can be mounted from numerous locations, such as, for example, stage 330 or a free-standing mount, so as to be positioned over second mirror 454. Detector 430 is shown as a CCD camera, though various embodiments of integrated bioanalysis system 500 of FIG. 2A may use a variety of detectors as previously described. Light emitted from conduits 110 of liquid processing manifold 100 is reflected from first mirror 452 to second mirror 454 to be detected by detector 430. If specificity of the wavelength of electromagnetic energy reaching detector 430 is indicated for some embodiments of integrated bioanalysis system 500, a filter 450 can be utilized in various embodiments the detection system 400. Additionally, control system 320 can control motor 456 for adjusting first mirror 452 and second mirror 454, as well as a motor or motors (not shown) for controlling the positioning of stage 330. Such control may be important not only for focusing the emitted light from liquid aliquot or slug 130 positioned at first end 114 of conduit 110, but for other functions, as will be discussed in more detail subsequently.

FIG. 2B is a cross-section of a side view depicting a liquid aliquot or slug 130 positioned at first end 114 of conduit 110 using the control of piston 140 and illuminated by excitation source 410, depicted as LEDs, though as previously described, capable of being a variety of devices. The light emitted by excited chemical or biochemical moieties in liquid aliquot or slug 130 is reflected from first mirror 452 and second mirror 454 to detector 430, as indicated by the hatched line. As previously discussed, the phrase “positioned at first end 114” in reference to position of liquid aliquot or slug 130 for the purpose of detection may include embodiments where liquid aliquot or slug 130 can be within the first end, and remains at a position proximal to first end 114, as well as embodiments where liquid aliquot or slug 130 can be at least partially extended from first end 114, as depicted in FIG. 2B. In some embodiments, liquid aliquot or slug 130 can be enveloped by an inert, immiscible fluid, such as an oil, for example a mineral oil, so that the protruding liquid is an oil droplet or film. Most importantly, liquid aliquot or slug 130 can be positioned at first end 114 so that it may be readily detected by detector 430.

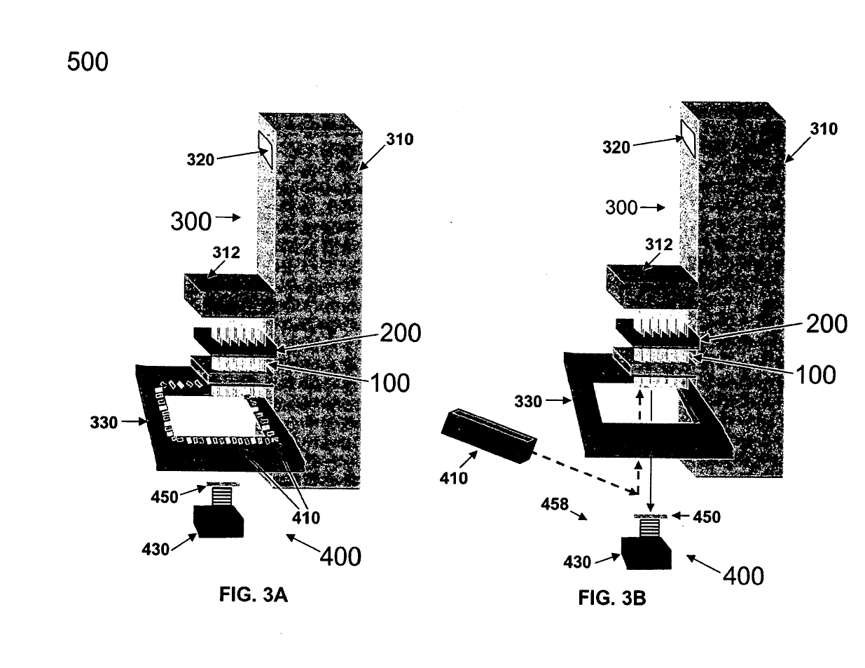

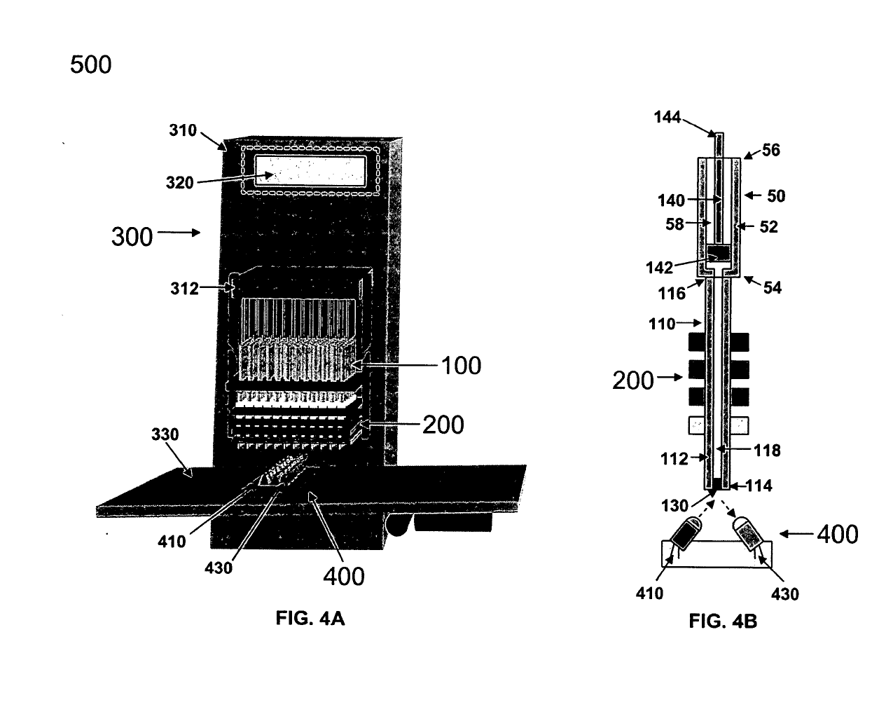

Additional designs of detection systems for integrated bioanalysis system 500 are illustrated by various embodiments of detection system 400 of FIG. 3A and FIG. 3B, as well as by various embodiments of detection system 400 of FIG. 4A and FIG. 4B. Various embodiments of detection system 400 of FIG. 3A utilize direct detection of light emitted from excited chemical or biochemical species in liquid aliquots or slugs 130 positioned at first ends 114 of conduit assembly 120 (see FIG. 1A and 1B) by positioning detector 430 directly in view of first ends 114. Various embodiments of detection system 400 indicated by FIG. 3B utilize a dichroic filter 458. Such filters can be selected to reflect light of specific wavelength range to excite chemical or biochemical moieties in liquid aliquot or slug 130 positioned at first end 114 of conduit 110, and then pass the emitted light from first end 114 to detector 430. In FIG. 4A, detection system 400 can be positioned on stage 330. In some embodiments of integrated bioanalysis system 500 of FIG. 4A, detection system 400 can be attached to stage 330, and stage 330 can move detection system 400 into position to detect all or a subset of first ends 114 of conduit assembly 120. In other embodiments of integrated bioanalysis system 500 of FIG. 4A, detection system 400 can be moved along stage 330 to position detection system 400 to detect all or a subset of the first ends 114 of conduit assembly 120.

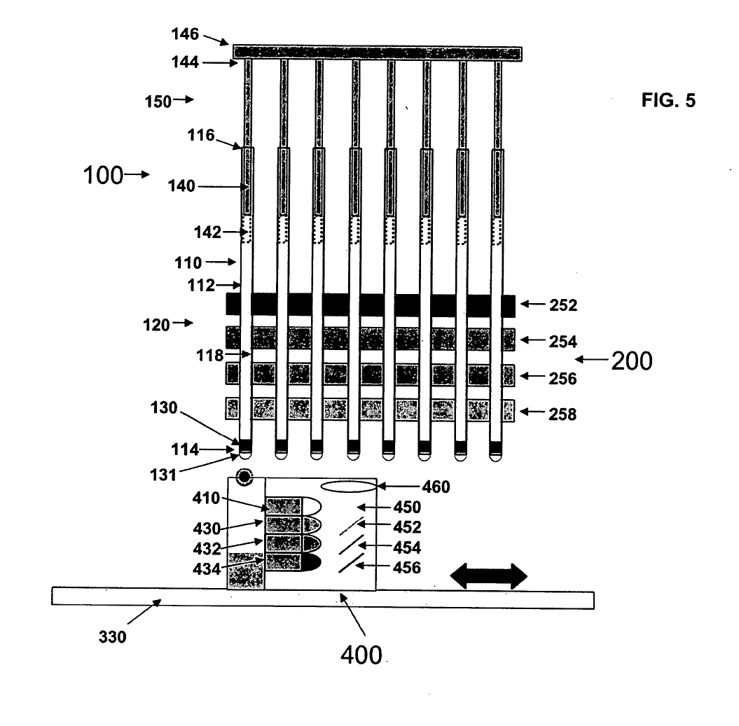

Various embodiments of detection system 400 of FIG. 4B utilize of two, three, or more LEDs, OLEDs, laser diodes, and the like that are positioned to illuminate all or a subset of the first ends 114 of conduit assembly 120 and have additionally two, three, or more detecting devices such as photodiodes, phototransistors, photoconductive detectors, linear sensor arrays, such as CMOS array detectors positioned to detect the light emitted by chemical or biochemical moieties in liquid aliquots or slugs 130 for all or a subset of first ends 114 of conduit assembly 120 (see FIG. 1A and 1B). Embodiments of integrated bioanalysis system 500 that can utilize various embodiments of detection system 400 of FIG. 5 are exemplary of a detection system that can be positioned and moved either along stage 330 or using stage 330. For some embodiments of a movable detection system 400 of FIG. 5 at least one excitation source, such as 430, 432, and 434, as well as at least one detector 410, and at least one dichroic filter, such as 450, 452, 454, and 456 can be used. Additionally, other optical elements, such as a focusing lens 460 may be incorporated in some embodiments of a movable detection system 400 of FIG. 5. An example of a detection system adaptable to embodiments of detection system 400 of FIG. 5 can be found in US 2006/0121602 (Hoshizaki, et al.; Jun. 8, 2006).

According to the various embodiments of a detection system 400 given in the above, such detection systems can comprise one or more excitation sources 410, such as LEDs, OLEDs, laser diodes, lasers, lamps, and the like, as well as one or more detectors 430, such as photodiodes, CCD detectors, and CMOS optical detectors, and the like. Additionally, optical systems may include operational amplifiers, and LED-current control circuits. Such components may have temperature dependent properties, meaning that their properties (e.g., LED intensity) can change with temperature variations. In that regard, variations of detection systems 400 for use with embodiments of integrated bioanalysis systems 500 may utilize a temperature compensation system that can, for example, maintain some or all of these components at a constant temperature to eliminate or reduce changes in the temperature dependent property or properties. The temperature dependent property may also include properties that are a derived or indirect function of a temperature dependent property. Thus, for example, if electrical resistance is a temperature dependent property, current or voltage, which would be functions of the resistance, could also be temperature dependent properties. Other temperature dependent properties may include, for example, temperature dependent properties of an optical detector, such as a photodiode. For example, the “dark current” or noise of a detector may be temperature dependent. Temperature sensors may thus include electronic circuits and signal measurement devices or elements configured to monitor, for example, dark current or noise.

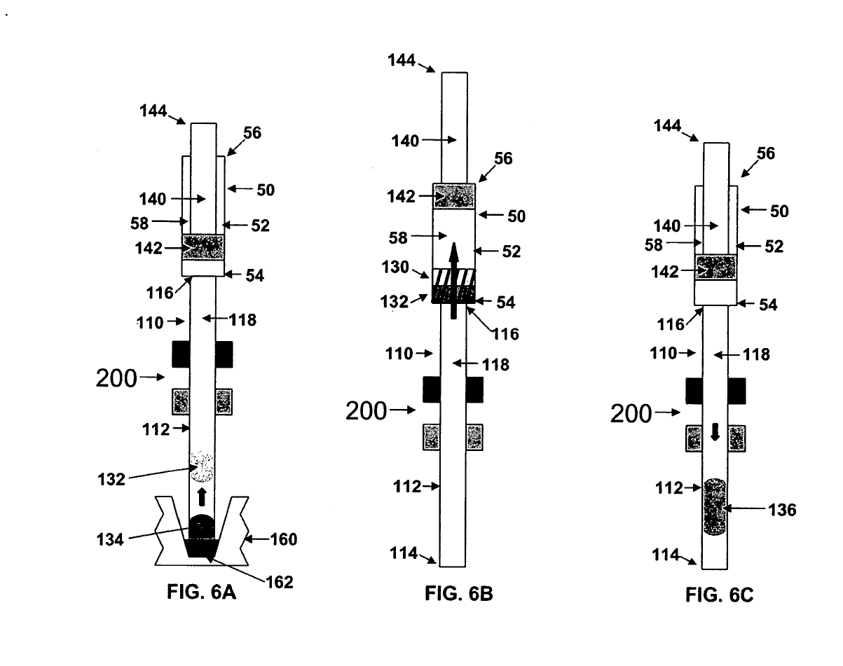

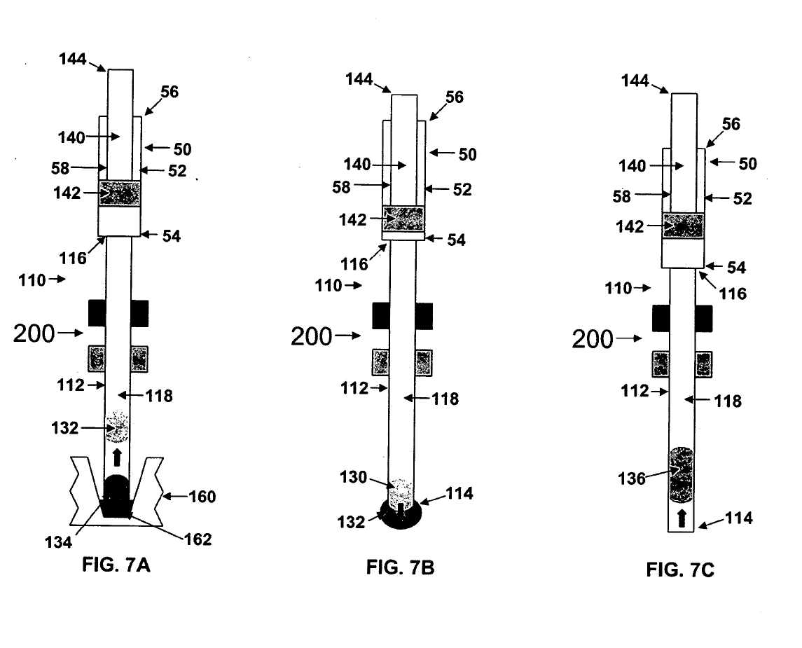

Liquid processing manifolds, such as various embodiments of disclosed liquid processing manifold 100, process liquids taken from samples and reagents held in containing means, for example, but not limited by microtiter plates, as well as various containers such as, but not limited by, vials, tubes, ampoules, and cuvettes, and the like, that are held in holders, such as racks. As one of ordinary skill in the art is apprised, many high-throughput bioanalyses are adapted to a microtiter plate format, for example based on a 8 by 12 array of wells, yielding 96 wells per plate, or higher orders of wells per plate based on a multiple of the 96 well pattern. In a typical operation, liquid processing manifold 100 is used primarily for the dispensing of fluids, while the bioanalysis steps of reacting and detecting are done in containing means. Mixing a reagent or reagents with a sample is necessary to the step of reacting. In that regard, various embodiments of methods for on-conduit mixing of a plurality of liquids using embodiments of liquid processing manifold 100, enabling on-conduit reactions thereby are depicted in FIGS. 6A, 6B, and 6C and FIGS. 7A, 7B, and 7C.

In various embodiments of a method depicted by FIGS. 6A, 6B, and 6C, first liquid slug 132 and second slug 134 can be drawn into conduit 110 from a containing means, such as 160, in which the sample or reagent, such as 162, has been dispensed (FIG. 6A). As depicted, first slug 132 and second slug 134 are separated by a segment of another fluid with which they are both immiscible, e.g., air. First slug 132 and second slug 134 can be drawn through conduit 110 and as depicted in FIG. 6B, into a second, wider bore, e.g., piston housing bore 58, using piston 140. In various embodiments of a method depicted by FIGS. 6A, 6B, and 6C, piston housing bore 58 has a diameter that is different than that of conduit bore 118. In FIGS. 6B, and 6C, as slugs 132 and 134 are drawn first into piston housing bore 58 and then moved back into conduit bore 118, they are mixed to form mixed slug 136. In various embodiments, the mixing of the first fluid and the second fluid can be increased by drawing mixed slug 136 into the second, wider bore and moving it back again into conduit bore 118. Other embodiments for a method of mixing a plurality of slugs based on the difference in bore diameter of a conduit, housing, or combination thereof, can utilize, for example, a tapered conduit, housing or combination thereof. Various embodiments of a method for mixing a plurality of liquid slugs depicted in FIGS. 7A, 7B, and 7C utilize the movement of liquid slugs between conduit bore 118 and first end 114 for on-conduit mixing of a plurality of liquid slugs. In FIG. 7A, a first liquid slug 132 can be drawn into conduit 110 from a containing means, such as 160, in which the sample or reagent, such as 162, has been dispensed. A second slug 134 can be drawn into first end 114 of conduit 110 as depicted in FIG. 7B. Using piston 140, first slug 132 and second slug 134 can be drawn up into conduit bore 118 as depicted in FIG. 7C, and then a portion of the combined first slug 132 and second slug 134 can be controllably exuded at first end 114 as depicted in FIG. 7B, effecting the mixing of first slug 132 and second slug 134 thereby to form mixed slug 136. Though various embodiments of the methods depicted by FIGS. 6A, 6B, and 6C and FIGS. 7A, 7B, and 7C have been demonstrated with a first and second slug, the variations of embodiments of the methods can be extended to mixing higher orders of liquid slugs for numerous samples and reagents. In addition to mixing, other benefits may be realized in the use of various embodiments of on-conduit manipulations of liquid aliquots or slugs. For example, sample preparation steps, such as, but not limited by, nucleic acid shearing may be done on-conduit.

As previously mentioned, an exemplary class of bioanalyses are enabled by a technique know as the polymerase chain reaction (PCR). One type of PCR reaction is known to those skilled in the art as real-time PCR, which has become a widely used in bioanalyses. An example of a system and method for real time PCR amplification can be found in U.S. Pat. No. 5,928,907 (Woudenberg, et al.; Jul. 27, 1999). A range of embodiments of real-time PCR methods can be performed using various embodiments of an integrated bioanalysis systems 500, as indicated by FIG. 4A, FIG. 4B and FIG. 5. In FIG. 5 conduit bore 118 can be at least partially filled with an oil, such as a mineral oil. Sample and reagents for conducting a quantitative PCR method have been mixed according to variations of methods for on-conduit mixing previously described, and can be formed as slug 130, which can be thermocycled, i.e., taken through a plurality of thermal cycles, using thermal system 200 for the purpose of amplification of targeted nucleic acid species.

Some embodiments of thermal system 200 of FIG. 5 can have between about 2 heating blocks to about 4 heating blocks, each of which are controlled to a targeted temperature to create a separate targeted heat zone in conduit 110. In some embodiments of a quantitative PCR method, a thermal setting of about 95° C. can be maintained for heating block 252, a thermal setting of about 109° C. can be maintained for heating block 254, a thermal setting of about 47° C. can be maintained for heating block 256, and a thermal setting of about 60° C. can be maintained for heating block 258. In some embodiments of apparatuses and methods for an integrated bioanalysis system 500, in order to decrease the cycle time, pairing heating blocks for the denaturation portion of the real-time PCR cycle and the extension/annealing portion of the real-time PCR cycle can be done. For example, for the denaturation portion of a PCR cycle, slug 130 can be moved into a thermal zone of about 109° C. of heating block 254 until the desired temperature for slug 130 of about 95° C. is reached, and then slug 130 can be moved into a thermal zone of about 95° C. of heating block 252 for the duration of the denaturation portion of the cycle. Similarly, during the extension/annealing portion of a PCR cycle, slug 130 can be moved into a thermal zone of about 47° C. of heating block 256 until the desired temperature for slug 130 of about 60° C. is reached, and then slug 130 can be moved into a thermal zone of about 60° C. of heating block 258 for the duration of the extension/annealing portion of the cycle. After each cycle, slug 130 is either in position at first end 114 for detection, or can be readily positioned at first end 114 for detection before the next cycle is initiated. As previously discussed, the phrase “positioned at first end 114” in reference to position of a liquid aliquot or slug 130 may include embodiments where liquid aliquot or slug 130 can be within the first end, and remains at a position proximal to first end 114, as well as embodiments where liquid aliquot or slug 130 can be at least partially extended from first end 114. In some embodiments, liquid aliquot or slug 130 can be enveloped by an inert, immiscible fluid, such as an oil, for example a mineral oil, so that the protruding liquid can be an oil droplet or film 131, as depicted in FIG. 5.

Though various embodiments of detection system 400 have been illustrated in various embodiments of figures presented, it is recognized by one of ordinary skill in the art that detection of slug 130 can be done on conduit 110 at a location other than the first end 114. For example, detection of slug 130 could be done in any location along conduit 110 using, for example, fiber optic cables both from an excitation source and to a detector.

The principles of luminescent detection in integrated bioanalysis systems have been described in connection with exemplary embodiments. Accordingly, it should be understood that these descriptions are made for the purpose of illustration, and are not intended to limit the scope of the disclosure. In that regard, what is disclosed herein is not intended to be exhaustive or to limit the illustrations and descriptions to the precise forms depicted. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalence.

What is claimed is:

1. A method for luminescent detection comprising:
providing a first conduit having a first end and a second end, said second end in fluid communication with fluid control means;
forming with the fluid control means a pendant drop at the first end of the first conduit;
selecting at least one excitation source, the at least one excitation source positioned proximal to the pendant drop, thereby creating at least one selected excitation source;
illuminating the pendant drop with the at least one selected excitation source to excite chemical or biochemical species present in the pendant drop; and
detecting with a detection system light emitted from excited chemical or biochemical species present in the pendant drop.

2. The method for luminescent detection of claim 1 wherein the light emitted is fluorescence.

3. The method for luminescent detection of claim 1 wherein the light emitted is phosphorescence.

4. The method for luminescent detection of claim 1 wherein the light emitted is chemiluminescence.

5. The method of luminescent detection of claim 1, further comprising:
thermocycling a liquid aliquot in the first conduit to produce chemical or biochemical species therein prior to forming the pendant drop with the liquid aliquot.

6. The method of luminescent detection of claim 1, further comprising:
amplifying targeted nucleic acid species within a liquid aliquot in the first conduit prior to forming the pendant drop with the liquid aliquot.

7. A method for mixing liquids in a conduit, the method comprising:
drawing a first liquid slug into a first conduit having a first bore;
drawing a second liquid slug into the first conduit having the first bore, such that the first and second liquid slugs are initially separated by a segment of a fluid that is immiscible with both the liquid of the first liquid slug and the liquid of the second liquid slug;
drawing the first and second liquid slug through the first bore into a second bore that is wider than the first bore until the first liquid slug and the second liquid slug contact each other and mix to form a third, mixed liquid slug; and
moving the third, mixed liquid slug into the first bore.

8. The method of claim 7, wherein the second bore is in the first conduit.

9. The method of claim 7, wherein the second bore is in a piston housing coupled to the first conduit.

10. The method of claim 7, further comprising:
drawing the third, mixed liquid slug into the second bore and subsequently moving the third, mixed liquid slug into the first bore.

11. An apparatus comprising:
a first conduit having a first end and a second end, the second end in fluid communication with fluid control means, wherein the fluid control means is capable of forming a pendant drop at the first end of the first conduit;
at least one excitation source proximal to the first end of the first conduit, wherein the pendant drop at the first end of the first conduit is illuminated by the excitation source; and
a detection system, wherein light emitted from the pendant drop is detected by the detection system.
